SEQUENCE LISTING (10 sequences)

SEQ ID NO: 1 - protein, 7 aa, Artificial Sequence
  HCDR3 of Anti-PD-1
RNIIVDY

SEQ ID NO: 2 - protein, 15 aa, Artificial Sequence
  HCDR2 of Anti-PD-1
LIATYITHYADFVKG

SEQ ID NO: 3 - protein, 10 aa, Artificial Sequence
  HCDR1 of Anti-PD-1
DSIDSLVNMG

SEQ ID NO: 4 - protein, 12 aa, Artificial Sequence
  HCDR3 of Anti-LAG-3
THYYTHRGPFDY

SEQ ID NO: 5 - protein, 17 aa, Artificial Sequence
  HCDR2 of Anti-LAG-3
AIHWTSSVTDYADSVYG

SEQ ID NO: 6 - protein, 10 aa, Artificial Sequence
  HCDR1 of Anti-LAG-3
GLTLSQYTMG

SEQ ID NO: 7 - protein, 114 aa, Artificial Sequence
  VHH of anti-PD-1 binding domain
QVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVSS

SEQ ID NO: 8 - protein, 121 aa, Artificial Sequence
  VHH of anti-LAG-3 binding domain
QVQLVESGGGVVQPGGSLRLSCAASGLTLSQYTMGWFRQAPGKERELVAAIHWTSSVTDYADSVYGRFTISRDDSKNTGYLQMNSLRAEDTAVYYCAATHYYTHRGPFDYWGQGTLVTVSS

SEQ ID NO: 9 - protein, 36 aa, Artificial Sequence
  Linker
GGGGGSGGGGSGGGGSGGGGSGGGGSGGGGSGGGGS

SEQ ID NO: 10 - protein, 498 aa, Artificial Sequence
  Full-length sequence
QVQLVESGGGVVQPGGSLRLSCAASDSIDSLVNMGWYRQAPGKQRELVALIATYITHYADFVKGRFTISRDNSKNTLYLQMNSLRAEDTAVYYCYARNIIVDYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSGGGGSGGGGSGGGGSQVQLVESGGGVVQPGGSLRLSCAASGLTLSQYTMGWFRQAPGKERELVAAIHWTSSVTDYADSVYGRFTISRDDSKNTGYLQMNSLRAEDTAVYYCAATHYYTHRGPFDYWGQGTLVTVSS

(19) United States
(12) Patent Application Publication
(10) Pub. No.: US 2022/0213192 A1
(43) Pub. Date: Jul. 7, 2022

(54) BISPECIFIC ANTIBODIES AGAINST PD-1 AND LAG-3

(71) Applicant: WUXI BIOLOGICS IRELAND LIMITED, Dublin (IE)

(72) Inventors: Qiong WU, Shanghai (CN); Yong ZHENG, Shanghai (CN); Yunying CHEN, Shanghai (CN); Jing LI, Shanghai (CN)

(21) Appl. No.: 17/606,744

(22) PCT Filed: Apr. 24, 2020
(86) PCT No.: PCT/CN2020/086830

§ 371 Date: Oct. 26, 2021

(30) Foreign Application Priority Data
Apr. 26, 2019 (CN) PCT/CN2019/084471

(51) Int. Cl. C07K 16/28; A61P 35/00; A61P 31/00

(52) U.S. Cl. CPC C07K 16/2818; A61K 2039/505; A61P 31/00; A61P 35/00

(57) ABSTRACT

Bispecific antibodies comprising a first targeting moiety which specifically binds to PD-1 and a second targeting moiety which specifically binds to LAG-3, wherein the first targeting moiety comprises a first VHH domain and the second targeting moiety comprises a second VHH domain. Amino acid sequences of the antibodies of the invention, cloning or expression vectors, host cells and methods for expressing or isolating the antibodies. Therapeutic compositions comprising the antibodies of the invention are also provided and methods for treating cancers and other diseases with the bispecific antibodies.

### TECHNICAL FIELD

The present invention relates to bispecific antibodies comprising a first targeting moiety which specifically binds to PD-1 and a second targeting moiety which specifically binds to LAG-3, wherein the first targeting moiety comprises a first VHH domain and the second targeting moiety comprises a second VHH domain. Moreover, the invention provides a polynucleotide encoding the antibodies, a vector comprising said polynucleotide, a host cell, a process for the production of the antibodies and immunotherapy in the treatment of cancer, infections or other human diseases using the bispecific antibodies.

### BACKGROUND OF THE INVENTION

Over the last few years, immunotherapy has evolved into a very promising new frontier for fighting some types of cancers. PD-1, one of the immune-checkpoint proteins, is an inhibitory member of CD28 family expressed on activated CD4+ T cells and CD8+ T cells as well as on B cell. PD-1 plays a major role in down-regulating the immune system.

PD-1 is a type I transmembrane protein and the structure consists of an immunoglobulin variable-like extracellular domain and a cytoplasmic domain containing an immunoreceptor tyrosine-based inhibitory motif (ITIM) and an immunoreceptor tyrosine-based switch motif (ITSM).

PD-1 has two known ligands, PD-L1 and PD-L2, which are cell surface expressed members of the B7 family. Upon ligation with its physiological ligand, PD-1 suppresses T-cell activation by recruiting SHP-2, which dephosphorylates and inactivates Zap70, a major integrator of T-cell receptor (TCR)-mediated signaling. As a result, PD-1 inhibits T cell proliferation and T cell functions such as cytokine production and cytotoxic activity.

Monoclonal antibodies targeting PD-1 can block PD-1/PD-L1 binding and boost the immune response against cancer cells. These drugs have shown a great deal of promise in treating certain cancers. Multiple approved therapeutic antibodies targeting PD-1 have been developed by several pharmaceutical companies, including Pembrolizumab (Keytruda), Nivolumab (Opdivo), Cemiplimab (Libtayo). These drugs have been shown to be effective in treating various types of cancer, including melanoma of the skin, non-small cell lung cancer, kidney cancer, bladder cancer, head and neck cancers, and Hodgkin lymphoma. They are also being studied for use against many other types of cancer.

Lymphocyte-activation gene 3, also known as LAG-3, is a type I transmembrane protein that is a member of the immune-globulin superfamily (IgSF). LAG-3 is a cell surface molecule expressed on activated T cells, NK cells, B cells and plasmacytoid dendritic cells, but not on resting T cells. LAG-3 shares approximately 20% amino acid sequence homology with CD4, but binds to MHC class II with higher affinity, providing negative regulation of T cell receptor signaling.

Blockade of LAG-3 in vitro augments T cell proliferation and cytokine production, and LAG-3-deficient mice have a defect in the downregulation of T cell responses induced by the superantigen staphylococcal enterotoxin B, by peptides or by Sendai virus infection. LAG-3 is expressed on both activated natural Treg (nTreg) and induced CD4+FoxP3+ Treg (iTreg) cells, where expression levels are higher than that observed on activated effector CD4+ T cells. Blockade of LAG-3 on Treg cells abrogates Treg cell suppressor function whereas ectopic expression of LAG-3 in non-Treg CD4+ T cells confers suppressive activity. On the basis of the immunomodulatory role of LAG-3 on T cell function in chronic infection and cancer, the predicted mechanism of action for LAG-3-specific monoclonal antibodies is to inhibit the negative regulation of tumor-specific effector T cells. Furthermore, dual blockade of the PD-1 pathway and LAG-3 has been shown in mice and human to be more effective for anti-tumor immunity than blocking either molecule alone.

Co-expression of LAG-3 and PD-1 was found on antigen-specific CD8+ T cells, and co-blockade of both lead to improved proliferation and cytokine production. Anti-LAG-3 in combination with anti-PD-1 therapy has entered clinical trials for various types of solid tumors.

### SUMMARY OF THE INVENTION

The present invention provides isolated antibodies, in particular bispecific antibodies.

In one aspect, the present invention provides a bispecific antibody or an antigen binding fragment thereof, comprising a first targeting moiety which specifically binds to human PD-1 and a second targeting moiety which binds to human LAG-3, wherein the first targeting moiety comprises a first VHH domain and the second targeting moiety comprises a second VHH domain.

In one embodiment, the aforesaid antibody or the antigen binding-fragment, the first targeting moiety binds to murine PD-1, the second targeting moiety binds to murine LAG-3.

In one embodiment, the present invention provides an antibody or an antigen binding fragment thereof, wherein the first VHH domain comprises H-CDR1, H-CDR2 and H-CDR3; wherein the H-CDR3 comprises a sequence as depicted in SEQ ID NO:1, and conservative modifications thereof; the H-CDR2 comprises a sequence as depicted in SEQ ID NO: 2, and conservative modifications thereof; the H-CDR1 comprises a sequence as depicted in SEQ ID NO:3, and conservative modifications thereof.

In one embodiment, the present invention provides an antibody or an antigen binding fragment thereof, wherein the second VHH domain comprises H-CDR1, H-CDR2, H-CDR3; wherein the H-CDR3 comprises a sequence as depicted in SEQ ID NO: 4, and conservative modifications thereof; the H-CDR2 comprises a sequence as depicted in SEQ ID NO: 5, and conservative modifications thereof; the H-CDR1 comprises a sequence as depicted in SEQ ID NO: 6, and conservative modifications thereof.

In one embodiment, the present invention provides an antibody or an antigen binding fragment thereof, wherein the first VHH domain comprises a sequence that is at least 70%, 80%, 85%, 90%, 95% or 99% homologous to SEQ ID NO: 7.

In one embodiment, the present invention provides an antibody or an antigen binding fragment thereof, wherein the second VHH domain comprises a sequence that is at least 70%, 80%, 85%, 90%, 95% or 99% homologous to SEQ ID NO: 8.

In one embodiment, the present invention provides an antibody or an antigen binding fragment thereof, wherein the first VHH domain comprises a sequence of SEQ ID NO: 7, and the second VHH domain comprises a sequence of SEQ ID NO: 8.

In one embodiment, the first VHH domain and the second VHH domain are linked by a peptide sequence, wherein the peptide sequence comprises

(a) a IgG Fc fragment comprising hinge region, CH2 and CH3, and/or

(b) a linker.

In one embodiment, the linker comprises a sequence of SEQ ID NO: 9.

In one embodiment, the present invention provides an antibody or an antigen binding fragment thereof, comprising a sequence of SEQ ID NO: 10.

The aforesaid antibody or an antigen binding fragment thereof, wherein the antibody or the antigen binding-fragment

a) binds to human PD-1 with a K_{D} of 2.92E-09 or less; and

b) binds to human LAG-3 with a KD of 3.01E-10 or less.

The sequence of said antibody is shown in Table 1 and Sequence Listing. The format of W3659-U14T4.G1-1.uIgG4.SP is VHH (anti-PD-1) -hinge-CH2-CH3-linker-VHH (anti-LAG-3), wherein the hinge-CH2-CH3 is a Fc fragment of IgG4.

TABLE 1
Deduced amino acid sequences of the antibodies

| Clone ID | | SEQ ID NO | Amino acid sequence |
|---|---|---|---|
| W3659-U1 | VHH of | 7 | QVQLVESGGGVVQPGGSLRLSCAASDSIDSLVNMGWYRQAP |
| 4T4.G1-1.u | anti-PD-1 | | GKQRELVALIATYITHYADFVKGRFTISRDNSKNTLYLQMN |
| IgG4.SP | binding | | SLRAEDTAVYYCYARNIIVDYWGQGTLVTVSS |
| | domain | | |
| | VHH of | 8 | QVLQVESGGGVVQPGGSLRLSCAASGLTLSQYTMGWFRQAP |
| | anti-LAG-3 | | GKERELVAAIHWTSSVTDYADSVYGRFTISRDDSKNTGYLQ |
| | binding | | MNSLRAEDTAVYYCAATHYYTHRGPFDYWGQGTLVTVSS |
| | domain | | |
| | Linker | 9 | GGGGGSGGGGSGGGGSGGGGSGGGGSGGGGSGGGGS |
| | Full-length | 10 | QVLQVESGGGVVQPGGSLRLSCAASDSIDSLVNMGWYRQAP |
| | sequence | | GKQRELVALIATYITHYADFVKGRFTISRDNSKNTLYLQMN |
| | | | SLRAEDTAVYYCYARNIIVDYWGQGTLVTVSSESKYGPPCP |
| | | | PCPAPEFLGGPSVFLFPPKPKDTLMISRPTEVTCVVVDVSQ |
| | | | EDPEVQFNWYVDGVEVHNAKTKPREEQFNSTYRVVSVLTVL |
| | | | HQDWLNGKEYKCKVSNKGLPSSIEKTISKAKGQPREPQVYT |
| | | | LPPSQEEMTKNQVSLTCLVKGFYPSDIAVEWESNGQPENNY |
| | | | KTTPPVLDSDGSFFLYSRLTVDKSRWQEGNVFSCSVMHEAL |
| | | | HNHYTQKSLSLSLGGGGGSGGGGSGGGGSGGGGSGGGGSGG |
| | | | GGSGGGGSQVQLVESGGGVVQPGGSLRLSCAASGLTLSQYT |
| | | | MGWFRQAPGKERELVAAIHWTSSVTDYADSVYGRFTISRDD |
| | | | SKNTGLYQMNSLRAEDTAVYYCAATHYYTHRGPFDYWGQGT |
| | | | LVTVSS |

The CDR sequences of said antibodies are shown in Table 2 and Sequence Listing.

TABLE 2
The CDR sequences of the antibodies

| | | SEQ | Amino acid |
|---|---|---|---|
| W3659-U14T4.G1-1.uIgG4.SP | | ID NO | sequence |
| Anti-PD-1 | Anti-PD-1: HCDR3 | 1 | RNIIVDY |
| binding | Anti-PD-1: HCDR3 | 2 | LIATYITHYADFVKG |
| domain | Anti-PD-1: HCDR3 | 3 | DSIDSLVNMG |
| Anti-LAG-3 | Anti-LAG-3: HCDR3 | 4 | THYYTHRGPFDY |
| binding | Anti-LAG-3: HCDR2 | 5 | AIHWTSSVTDYADSVYG |
| domain | Anti-LAG-3: HCDR1 | 6 | GLTLSQYTMG |

The antibody of the invention can be a chimeric antibody.

The antibody of the invention can be a humanized antibody, or a fully human antibody.

The antibody of the invention can be a rodent antibody.

In a further aspect, the invention provides a nucleic acid molecule encoding the antibody, or antigen binding fragment thereof.

The invention provides a cloning or expression vector comprising the nucleic acid molecule encoding the antibody, or antigen binding fragment thereof.

The invention also provides a host cell comprising one or more cloning or expression vectors.

In yet another aspect, the invention provides a process, comprising culturing the host cell of the invention and isolating the antibody.

In a further aspect, the invention provides pharmaceutical composition comprising the antibody, or the antigen binding fragment of said antibody in the invention, and one or more of a pharmaceutically acceptable excipient, a diluent or a carrier.

The invention provides an immunoconjugate comprising said antibody, or antigen-binding fragment thereof in this invention, linked to a therapeutic agent.

Wherein, the invention provides a pharmaceutical composition comprising said immunoconjugate and one or more of a pharmaceutically acceptable excipient, a diluent or a carrier.

The invention also provides a method of modulating an immune response in a subject comprising administering to the subject the antibody, or antigen binding fragment of any one of said antibodies in this invention.

The invention also provides the use of said antibody or the antigen binding fragment thereof in the manufacture of a medicament for the treatment or prophylaxis of an immune disorder or cancer.

The invention also provides a method of inhibiting growth of tumor cells in a subject, comprising administering to the subject a therapeutically effective amount of said antibody, or said antigen-binding fragment to inhibit growth of the tumor cells.

Wherein, the invention provides the method, wherein the tumor cells are of a cancer selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

### The Features and Advantages of this Invention

A bispecific antibody against both PD-1 and LAG-3 pathways may provide several benefits in cancer therapy. Compared with anti-PD-1 therapy, the bispecific antibody may increase the response rate on PD-1 and LAG-3 double positive cancers.

### BRIEF DESCRIPTION OF THE DRAWINGS

FIG. 1 shows PD-1×LAG-3 bispecific antibodies bind to human PD-1 protein.

FIG. 2 shows PD-1×LAG-3 bispecific antibodies to human LAG-3 protein.

FIG. 3 shows PD-1×LAG-3 bispecific antibodies to mouse PD-1 protein.

FIG. 4 shows PD-1×LAG-3 bispecific antibodies to mouse LAG-3 protein.

FIG. 5 shows PD-1×LAG-3 bispecific antibodies to cell surface cynomolgus PD-1.

FIG. 6 shows PD-1×LAG-3 bispecific antibodies to cynomolgus LAG-3 protein.

FIG. 7 shows the binding of PD-1×LAG-3 bispecific antibodies to human CTLA-4, CD28 and CD4 protein. FIG. 7A shows PD-1×LAG-3 bispecific antibodies do not bind to human CTLA-4 protein; FIG. 7B shows PD-1×LAG-3 bispecific antibodies do not bind to human CD28 protein; FIG. 7C shows PD-1×LAG-3 bispecific antibodies do not bind to human CD4 protein

FIG. 8 shows PD-1×LAG-3 bispecific antibodies bind to human PD-1 and LAG-3 protein simultaneously.

FIG. 9 shows PD-1×LAG-3 bispecific antibodies block the binding of PD-1 to PD-L1 expressing cells.

FIG. 10 shows PD-1×LAG-3 bispecific antibodies block the binding of LAG-3 to MHC-II on Raji cells.

FIG. 11 shows PD-1×LAG-3 bispecific antibodies enhance NFAT pathways in PD-1 expressing Jurkat.

FIG. 12 shows PD-1×LAG-3 bispecific antibodies enhance IL-2 pathways in LAG-3 expressing Jurkat.

FIG. 13 shows PD-1×LAG-3 bispecific antibodies enhance NFAT pathways in LAG-3 and PD-1 expressing Jurkat.

FIG. 14 shows the effects of PD-1×LAG-3 bispecific antibodies on human allogeneic mixed lymphocyte reaction (MLR). FIG. 14A shows PD-1×LAG-3 bispecific antibodies enhance IL-2 production in MLR assay. FIG. 14B shows PD-1×LAG-3 bispecific antibodies enhance IFN-γ production in MLR assay.

FIG. 15 shows PD-1×LAG-3 bispecific antibodies enhance IL-2 production of PBMC stimulated with SEB.

FIG. 16 shows W3659-U14T4.G1-1.uIgG4.SP was stable in fresh human serum for up to 14 days.

FIG. 17 shows the effect of PD-1×LAG-3 bispecific antibodies on tumor in mice. FIG. 17A shows PD-1×LAG-3 bispecific antibodies inhibit the growth of colon26 tumor in mice. FIG. 17B shows Survive curves of treated mice. FIG. 17C shows weight changes of treated mice.

### DETAILED DESCRIPTION

In order that the present invention may be more readily understood, certain terms are first defined. Additional definitions are set forth throughout the detailed description.

The terms “Programmed Death 1”, “Programmed Cell Death 1”, “Protein PD-1”, “PD-1”, “PD1”, “PDCD1”, “hPD-1”, “CD279” and “hPD-F” are used interchangeably, and include variants, isoforms, species homologs of human PD-1, PD-1 of other species, and analogs having at least one common epitope with PD-1.

The term “antibody” as referred to herein includes whole antibodies and any antigen-binding fragment (i.e., “antigen-binding portion”) or single chains thereof. An “antibody” refers to a protein comprising at least two heavy (H) chains and two light (L) chains inter-connected by disulfide bonds, or an antigen-binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The CDRs in heavy chain are abbreviated as H-CDRs, for example H-CDR1, H-CDR2, H-CDR3, and the CDRs in light chain are abbreviated as L-CDRs, for example L-CDR1, L-CDR2, L-CDR3.

The term “antibody” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. The term “antibody” also includes antibody fragments such as scFv, dAb, bispecific antibodies comprising a first VHH domain and a second VHH domain, and other antibody fragments that retain antigen-binding function, i.e., the ability to bind PD-1 and LAG-3 specifically. Typically, such fragments would comprise an antigen-binding fragment.

An antigen-binding fragment typically comprises an antibody light chain variable region (VL) and an antibody heavy chain variable region (VH), however, it does not necessarily have to comprise both. For example, a so-called Fd antibody fragment consists only of a VH domain and CH1 domain, but still retains some antigen-binding function of the intact antibody.

The term “cross-reactivity” refers to binding of an antigen fragment described herein to the same target molecule in human, monkey, and/or murine (mouse or rat). Thus, “cross-reactivity” is to be understood as an interspecies reactivity to the same molecule X expressed in different species, but not to a molecule other than X. Cross-species specificity of a monoclonal antibody recognizing e.g. human PD-1, to monkey, and/or to a murine (mouse or rat) PD-1, can be determined, for instance, by FACS analysis.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The terms “treatment” and “therapeutic method” refer to both therapeutic treatment and prophylactic/preventative measures. Those in need of treatment may include individuals already having a particular medical disorder as well as those who may ultimately acquire the disorder.

The terms “conservative modifications” i.e., nucleotide and amino acid sequence modifications which do not significantly affect or alter the binding characteristics of the antibody encoded by the nucleotide sequence or containing the amino acid sequence. Such conservative sequence modifications include nucleotide and amino acid substitutions, additions and deletions. Modifications can be introduced into the sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The terms “LAG-3”, “lymphocyte-activation gene 3”, “CD223” are used interchangeably, and include variants, isoforms, species homologs of human LAG-3, LAG-3 of other species, and analogs having at least one common epitope with LAG-3.

The terms “single domain antibody”, “heavy chain antibody”, “HCAb” are used interchangeably, refers to an antibody that contains two VH domains and no light chains. Heavy chain antibodies were originally derived from Camelidae (camels, dromedaries, and llamas). Although devoid of light chains, HCAbs have an authentic antigen-binding repertoire. The variable domain of a heavy chain antibody (VHH domain) represents the smallest known antigen-binding unit generated by adaptive immune responses. The terms “VHH” refers to variable domain of the heavy chain of HCAb.

The term “homolog” and “homologous” as used herein are interchangeable and refer to nucleic acid sequences (or its complementary strand) or amino acid sequences that have sequence identity of at least 70% (e.g., at least 70%, 75%, 80%, 85%, 88%, 90%, 91%, 92%, 93%, 94%, 95%, 96%, 97%, 98%, 99%) to another sequences when optimally aligned.

### EXAMPLES

### Example 1: Research Materials Preparation

### 1. Commercial Materials

| Company | Vendor | Cat. |
|---|---|---|
| Factor Xa protease | New England Biolabs | P8010L |
| Protein A column | GE Healthcare | 175438 |
| Lipofactamine | Invitrogen | 11668019 |
| Jurkat cell | ATCC | TIB-152 |
| Raji cell | ATCC | CCL-86 |
| HPR-anti-mouse IgG | Bethyl | A90-231P |
| PE-anti-human IgG | Jackson Laboratory | 109-115-098 |
| SEE | Toxin Technology | ET404 |
| Recombinant human CD4 protein | Sino Biological | 0400-H08H-5 |
| Cynomolgus LAG-3 (His tag) | Sino Biological | 90841-C08H |
| Mouse PD-1 (His tag) | Sino Biological | 50124-M08H |
| Human PD-1 (His tag) | Sino Biological | 10377-H08H |
| Cynomolgus PD-1 (His tag) | AcrobioSystems | PD-1-C5223 |
| One-Glo | Promega | E6130 |
| FreeStyle 293 Expression Medium | Invitrogen | 12338018 |
| Ficoll | Stemcell | 07851 |
| Human CD4⁺ T cell enrichment kit | Stemcell | 19052 |
| Human monocyte enrichment kit | Stemcell | 19059 |
| IL-2 standard | R&D | 202-IL-010 |
| IL-2 capture antibody | R&D | MAB602 |
| IL-2 detection antibody | R&D | BAF202 |
| IFN-γ standard | PeproTech | 300-02 |
| IFN-γ capture antibody | Pierce | M700A |
| IFN-γ detection antibody | Pierce | M701B |
| Blasticidin | Invitrogen | A1113902 |
| Hygromycin | Invitrogen | 10687010 |

### 2. Antigen and Other Proteins Generation

### 2.1 Production of Antigens

Nucleic acid encoding human PD-1, mouse PD-1, human LAG-3, mouse LAG-3 and cynomolgus LAG-3 ECD (extracellular domain) were synthesized by Sangon Biotech. PD-1 or LAG-3 gene fragments were amplified from the synthesized nucleic acid and inserted into the expression vector pcDNA3.3 (ThermoFisher). The inserted PD-1 or LAG-3 gene fragment was further confirmed by DNA sequencing. Fusion proteins containing human LAG-3 ECD with various tags, including human Fc, mouse Fc, were obtained by transfection of human PD-1 or LAG-3 gene into 293F cells (ThermoFisher). The cells were cultured in FreeStyle 293 Expression Medium at 37° C., 5% CO₂. After 5 days of culture, supernatants harvested from the culture of transiently transfected cells were used for protein purification. The fusion proteins were purified by protein A and/or SEC column. An untagged LAG-3 ECD protein was generated by cleavage of ECD-hFc fusion protein with a cut site using Factor Xa protease. Purified proteins were used for screening and characterization.

Mouse Fc-tagged human PD-L1 ECD, human CTLA-4 ECD and CD28 ECD were generated as above.

### 2.2 Production of Benchmark Antibodies

Gene sequences of anti-human PD-1 or LAG-3 benchmark antibodies (W339-BMK1 and W305-BMK1) were synthesized based on the information disclosed in patent applications US20110150892A1 (W339-BMK1 was referred to as “25F7”) and WO2006121168 (W305-BMK1 was referred to as “5C4”), respectively.

Sequences of anti-human PD-1×LAG-3 benchmark antibodies W365-BMK1, W365-BMK2 and W365-BMK3 were synthesized in based on the information disclosed in patent applications WO2015200119A8 (W365-BMK1 was referred to as “SEQ25 & SEQ27”), WO2017087589A2 (W365-BMK2 was referred to as “SEQ110”) and WO2015200119A8 (W365-BMK3 was referred to as “SEQ 5 and 4”), respectively. The synthesized gene sequences were incorporated into plasmids pcDNA3.3. The cells transfected with the plasmids were cultured for 5 days and supernatant was collected for protein purification using Protein A column. The obtained benchmark antibodies were analyzed by SDS-PAGE and SEC, and then stored at −80° C.

W3056-AP17R1-2H2-Z1-R1-14A1-Fc-V2 (3056, anti-PD-1) and W3396-P2R2(L)-1E1-z4-R2-2-Fc (3396, anti-LAG-3) were discovered in TAD department of Wuxi Biologics.

### 3. Cell Line Generation

Cynomolgus PD-1 transfectant cell line was generated. Briefly, 293F cells were transfected with pcDNA3.3 expression vector containing full-length of human, cynomolgus PD-1 using Lipofectamine transfection kit according to manufacturer's protocol, respectively. At 48-72 hours post transfection, the transfected cells were cultured in medium containing blasticidin for selection and tested for target expression.

Jurkat cell lines were transfected with plasmids containing human full length PD-1/NFAT reporter or LAG-3/IL-2 reporter using Nucleofactor (Lonza). At 72 hours post transfection, the transfected cells were cultured in medium containing hygromycin for selection and tested for target expression. Jurkat cells expressing human PD-1 or LAG-3 along with stably integrated NFAT or IL-2 luciferase reporter gene were obtained after two months.

### Example 2: Bispecific Antibody Generation

### 1. Construct Expression Vectors

The method for producing the first VHH binding PD-1 was described in PCT application No. PCT/CN2019/078515, and the method for producing the second VHH binding LAG-3 was described in PCT application No. PCT/CN2019/078315.

DNA sequences encoding the anti-PD-1 VHH and anti-LAG-3 VHH were synthesized by GENEWIZ (Suzhou, China). Then anti-PD-1 VHH and anti-LAG-3 VHH were subcloned at N-terminal and C-terminal of hinge region and IgG4 Fc region in the pYF expression vector respectively.

### 2. Small Scale Transfection, Expression and Purification

The plasmid of bispecific antibody was transfected into Expi293 cells. Cells were cultured for 5 days and supernatant was collected for protein purification using Protein A column (GE Healthcare). The obtained antibody was analyzed by SDS-PAGE and HPLC-SEC, and then stored at −80° C.

The purity of antibodies was determined by SEC-HPLC using Agilent 1260 Infinity HPLC. Antibody solution was injected on a TSKgel SuperSW3000 column using 50 mM sodium phosphate, 0.15 M NaCl, pH 7.0 buffer. The running time was 20 min. Peak retention times on the column were monitored at 280 nm. Data was analyzed using ChemStation software (V2.99.2.0).

### 3. Results

### Sequence of Lead Candidates

The sequences of antibody leads are listed in the Table 2 and the CDRs are listed in Table 1.

### Example 4: In Vitro Characterization

### 1. Binding of PD-1×LAG-3 Bispecific Antibodies to Human PD-1 or LAG-3 Protein

Plates were coated with of PD-1×LAG-3 antibodies overnight at 4° C. After blocking and washing, various concentrations of mouse Fc-tagged PD-1 protein or LAG-3 protein were added to the plates and incubated at room temperature for 1 hour. The plates were then washed and subsequently incubated with HRP-labeled goat anti-mouse IgG antibody for 1 hour. After washing, TMB substrate was added and the color reaction was stopped by 2M HCl. The absorbance at 450 nm was read using a microplate reader.

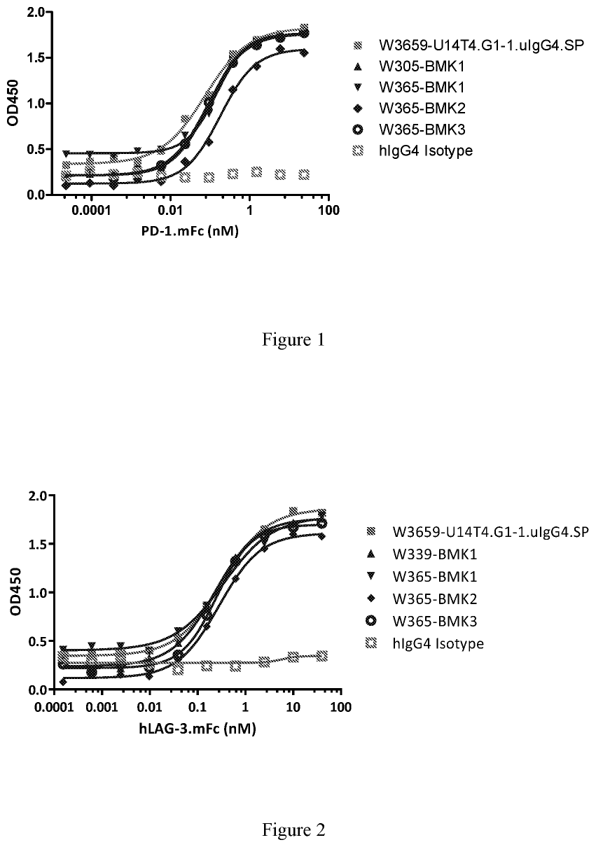

As shown in FIG. 1 and table 3, the EC₅₀ of W3659-U14T4.G1-1.uIgG4.SP for binding to PD-1 protein is comparable to the benchmarks.

TABLE 3
EC₅₀ of PD-1 × LAG-3 bispecific antibodies bind to human PD-1 protein

| Antibody | EC₅₀ (nM) |
|---|---|
| W3659-U14T4.G1-1.uIgG4.SP | 0.07 |
| W305-BMK1 | 0.09 |
| W365-BMK1 | 0.15 |
| W365-BMK2 | 0.18 |
| W365-BMK3 | 0.09 |

As shown in FIG. 2 and table 4, the EC₅₀ of W3659-U14T4.G1-1.uIgG4.SP for binding to LAG-3 protein is comparable to the benchmarks.

TABLE 4
EC₅₀ of PD-1 × LAG-3 bispecific antibodies bind to human LAG-3 protein

| Antibody | EC₅₀ (nM) |
|---|---|
| W3659-U14T4.G1-1.uIgG4.SP | 0.32 |
| W305-BMK1 | 0.23 |
| W365-BMK1 | 0.35 |
| W365-BMK2 | 0.28 |
| W365-BMK3 | 0.25 |

### 2. Binding of PD-1×LAG-3 Bispecific Antibodies to Mouse PD-1 or LAG-3

Plates were coated with of PD-1×LAG-3 antibodies overnight at 4° C. After blocking and washing, various concentrations of His-tagged mouse PD-1 or LAG-3 protein were added to the plates and incubated at room temperature for 1 hour. The plates were then washed and subsequently incubated with HRP-labeled goat anti-His IgG antibody for 1 hour. After washing, TMB substrate was added and the color reaction was stopped by 2M HCl. The absorbance at 450 nm was read using a microplate reader.

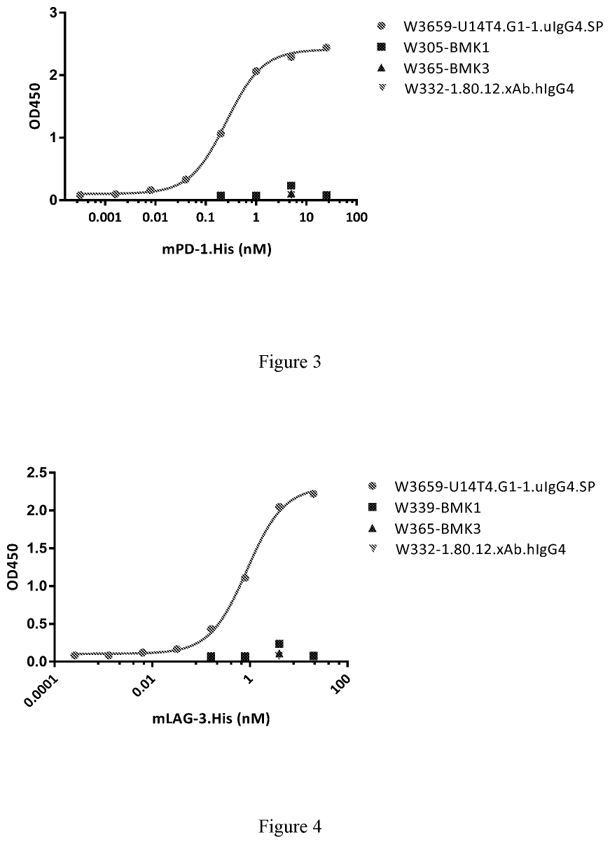

As shown in FIG. 3 and table 5, only W3659-U14T4.G1-1.uIgG4.SP, but not the BMKs, can bind to mouse PD-1 protein.

TABLE 5
EC₅₀ of PD-1 × LAG-3 bispecific antibodies bind to mouse PD-1 protein

| Antibody | EC₅₀ (nM) |
|---|---|
| W3659-U14T4.G1-1.uIgG4.SP | 0.26 |
| W305-BMK1 | Not Bind |
| W365-BMK3 | Not Bind |

As shown in FIG. 4 and table 6, only W3659-U14T4.G1-1.uIgG4.SP, but not the BMKs, can bind to mouse LAG-3 protein.

TABLE 6
EC₅₀ of PD-1 × LAG-3 bispecific antibodies bind to mouse PD-1 protein

| Antibody | EC₅₀ (nM) |
|---|---|
| W3659-U14T4.G1-1.uIgG4.SP | 0.87 |
| W305-BMK1 | Not Bind |
| W365-BMK3 | Not Bind |

### 3. Binding of PD-1×LAG-3 Bispecific Antibodies to Cynomolgus PD-1 or LAG-3

For cynomolgus PD-1, 293F cells expressing cynomolgus PD-1 were incubated with various concentrations of PD-1×LAG-3 antibodies, respectively. PE-labeled goat anti-human IgG antibody was used to detect the binding of PD-1×LAG-3 antibodies onto the cells. MFI of the cells was measured by flow cytometry and analyzed by FlowJo (version 7.6.1).

For cynomolgus LAG-3, plates were coated with of PD-1×LAG-3 antibodies overnight at 4° C. After blocking and washing, various concentrations of His-tagged cynomolgus LAG-3 were added to the plates and incubated at room temperature for 1 hour. The plates were then washed and subsequently incubated with HRP-labeled goat anti-His IgG antibody for 1 hour. After washing, TMB substrate was added and the color reaction was stopped by 2M HCl. The absorbance at 450 nm was read using a microplate reader.

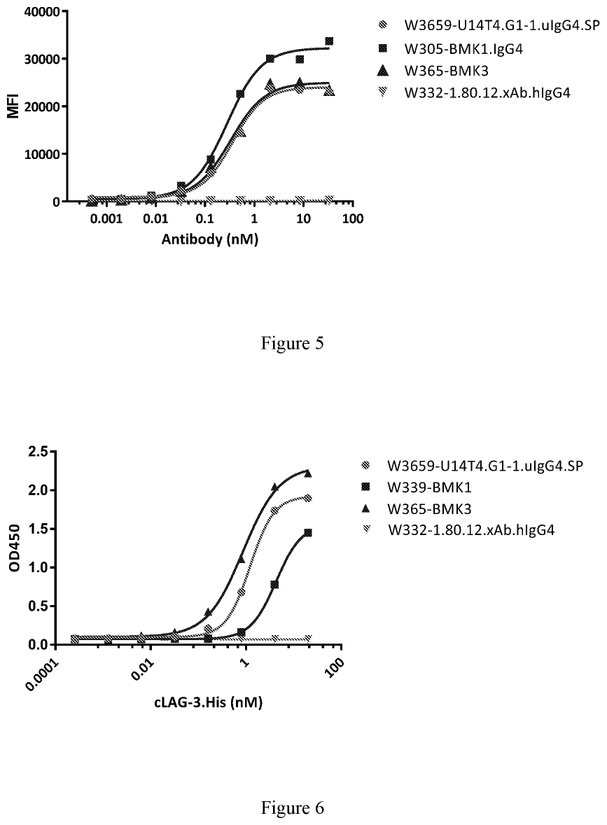

As shown in FIG. 5 and table 7, the EC₅₀ of W3659-U14T4.G1-1.uIgG4.SP for binding to LAG-3 protein is comparable to the BMKs.

TABLE 7
EC₅₀ of PD-1 × LAG-3 bispecific antibodies
bind to cell surface cynomolgus PD-1

| Antibody | EC₅₀ (nM) |
|---|---|
| W3659-U14T4.G1-1.uIgG4.SP | 0.36 |
| W305-BMK1 | 0.28 |
| W365-BMK3 | 0.33 |

As shown in FIG. 6 and table 8, the EC₅₀ of W3659-U14T4.G1-1.uIgG4.SP for binding to LAG-3 protein is comparable to the W365-BMK3 and better than W339-BMK1.

TABLE 8
EC₅₀ of PD-1 × LAG-3 bispecific antibodies
bind to cynomolgus LAG -3 protein

| Antibody | EC₅₀ (nM) |
|---|---|
| W3659-U14T4.G1-1.uIgG4.SP | 1.21 |
| W339-BMK1 | 4.27 |
| W365-BMK3 | 0.87 |

### 4. Cross-Reactivity to Human CD4, CTLA-4 and CD28

Cross-reactivity to human CD4, CTLA-4 or CD28 was measured by ELISA. Plates were coated with human CD4, CTLA-4 or CD28 at 1 μg/mL overnight at 4° C. After blocking and washing, various concentrations of PD-1×LAG-3 antibodies were added to the plates and incubated at room temperature for 1 h. The plates were then washed and subsequently incubated with corresponding secondary antibody for 60 min. After washing, TMB substrate was added and the color reaction was stopped by 2M HCl.

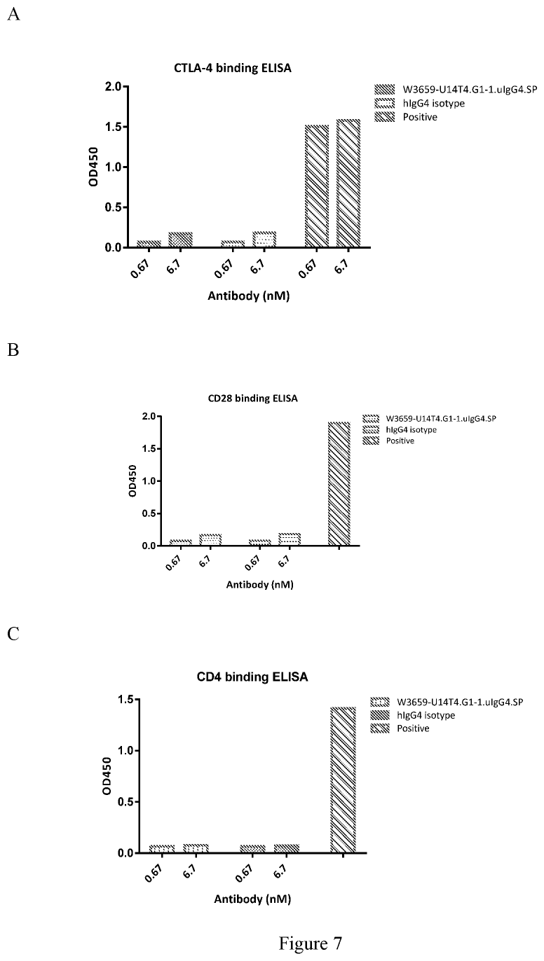

Results in FIG. 7A, 7B and 7C indicate that PD-1×LAG-3 bispecific antibodies did not bind to human CTLA-4, CD28 or CD4 protein.

### 5. Affinity Test Against Human, Mouse, Cynomolgus PD-1 and LAG-3 by SPR

Binding affinity of the bispecific antibodies to the antigen was determined by SPR assay using Biacore 8K. PD-1×LAG-3 antibodies were captured on an anti-human IgG Fc antibody immobilized CM5 sensor chip (GE). His tagged human PD-1 protein (MW: 40KD) and cynomolgus PD-1 (MW: 40KD) at different concentrations were injected over the sensor chip at a flow rate of 30 μL/min for an association phase of 120 s, followed by 800 s dissociation. His tagged mouse LAG-3 protein (MW: 45KD) at different concentrations were injected over the sensor chip at a flow rate of 30 μL/min for an association phase of 120 s, followed by 3600 s dissociation. His tagged mouse PD-1 protein (MW: 45KD) at different concentrations were injected over the sensor chip at a flow rate of 30 μL/min for an association phase of 60 s, followed by 90 s dissociation. The chip was regenerated by 10 mM glycine (pH 1.5) after each binding cycle.

For affinity against human LAG-3, PD-1×LAG-3 antibodies were immobilized on a CM5 sensor chip. Human LAG-3 without tag at different concentrations were injected over the sensor chip at a flow rate of 30 μL/min for an association phase of 180 s, followed by 3600 s dissociation using single-cycle kinetics method. The chip was regenerated with 10 mM glycine (pH 1.5).

The sensorgrams of blank surface and buffer channel were subtracted from the test sensorgrams. The experimental data was fitted by 1:1 model using Langmiur analysis.

TABLE 9
Affinity of PD-1 × LAG-3 bispecific antibodies against human, mouse and cynomolgus PD-1

| Antigen | Ab | ka (1/Ms) | kd (1/s) | KD (M) |
|---|---|---|---|---|
| Human PD-1 | W3659-U14T4.G1-1.uIgG4.SP | 2.52E+05 | 7.36E−04 | 2.92E−09 |
| | W305-BMK1 | 4.02E+05 | 1.35E−03 | 3.37E−09 |
| | W365-BMK3 | 3.80E+05 | 1.36E−03 | 3.58E−09 |
| Cynomolgus PD-1 | W3659-U14T4.G1-1.uIgG4.SP | 1.97E+05 | 1.66E−03 | 8.45E−09 |
| | W305-BMK1 | 3.51E+05 | 8.39E−04 | 2.39E−09 |
| | W365-BMK3 | 2.93E+05 | 6.74E−04 | 2.30E−09 |
| Mouse PD-1 | W3659-U14T4.G1-1.uIgG4.SP | 1.52E+05 | 4.99E−03 | 3.29E−08 |

TABLE 10
Affinity of PD-1 × LAG-3 bispecific antibodies against human and cynomolgus LAG-3

| Antigen | Ab | ka (1/Ms) | kd (1/s) | KD (M) |
|---|---|---|---|---|
| Human LAG-3 | W3659-U14T4.G1-1.uIgG4.SP | 3.00E+05 | 9.03E−05 | 3.01E−10 |
| | W339-BMK1 | 4.87E+05 | 3.34E−04 | 6.85E−10 |
| | W365-BMK3 | 1.02E+07 | 8.70E−04 | 8.51E−11 |
| Mouse LAG-3 | W3659-U14T4.G1-1.uIgG4.SP | 1.96E+06 | <1.00E−05 | <5.10E−12 |

### 6. Dual Binding of PD-1×LAG-3 Bispecific Antibodies to Human PD-1 and LAG-3 Protein

Plates were coated with mouse Fc-tagged human PD-1 at 1 μg/mL overnight at 4° C. After blocking and washing, various concentrations of PD-1×LAG-3 antibodies were added to the plates and incubated at room temperature for 1 hour after washing. The plates were then washed and subsequently incubated with His-tagged LAG-3 protein for 1 hour. After washing, HRP anti-His antibody was added to the plate and incubated at room temperature for 1 hour. After washing, TMB substrate was added and the color reaction was stopped by 2M HCl. The absorbance at 450 nm was read using a microplate reader.

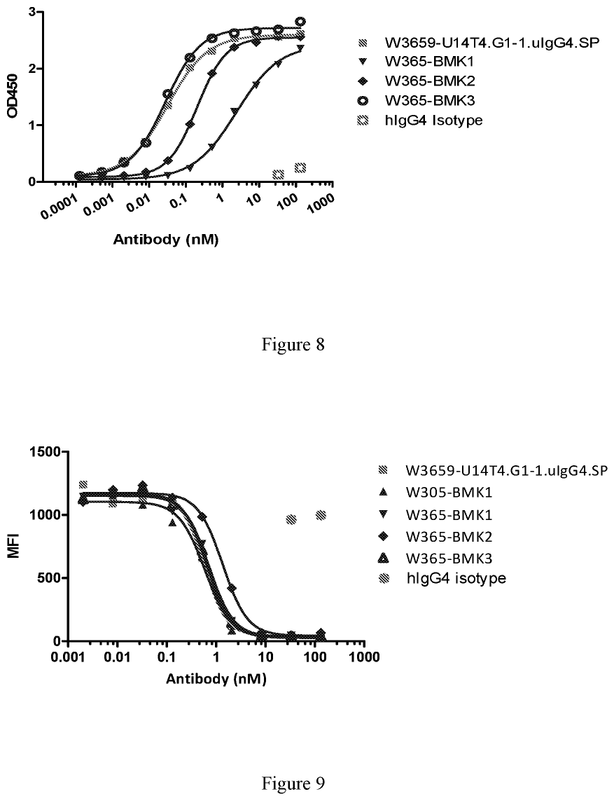

As shown in FIG. 8 and table 11, the EC50 of W3659-U14T4.G1-1.uIgG4.SP for binding to LAG-3 protein is comparable to the W365-BMK3 and better than W365-BMK1 and BMK2.

TABLE 11
EC₅₀ of PD-1 × LAG-3 bispecific antibodies
bind to human PD-1 and LAG-3 protein

| Antibody | EC₅₀ (nM) |
|---|---|
| W3659-U14T4.G1-1.uIgG4.SP | 0.03 |
| W365-BMK1 | 2.41 |
| W365-BMK2 | 0.2 |
| W365-BMK3 | 0.03 |

### 7. Blocking of PD-L1 Protein Binding to PD-1 Expressing Cells

Antibodies were serially diluted in 1% BSA-PBS and mixed with mFc-tagged PD-L1 protein at 4° C. The mixture was transferred into the 96-well plates seeded with PD-1 expressing CHO-S cells. Goat anti-mouse IgG Fc-PE antibody was used to detect the binding of PD-L1 protein to PD-1 expressing cells. The MFI was evaluated by flow cytometry and analyzed by the software FlowJo.

As shown in FIG. 9 and table 12, the EC₅₀ of W3659-U14T4.G1-1.uIgG4.SP for blocking the binding of PD-1 to PD-L1 expressed cells is comparable to the BMKs.

TABLE 12
EC₅₀ of PD-1 × LAG-3 bispecific antibodies
block the binding of PD-1 to PD-L1

| Antibody | EC₅₀ (nM) |
|---|---|
| W3659-U14T4.G1-1.uIgG4.SP | 0.58 |
| W305-BMK1 | 0.59 |
| W365-BMK1 | 0.72 |
| W365-BMK2 | 1.36 |
| W365-BMK3 | 0.64 |

### 8. Blocking of LAG-3 Protein Binding to MHC-II Expressed on Raji Cells

Antibodies were serially diluted in 1% BSA-PBS and incubated with mouse Fc-tagged LAG-3 protein at 4° C. The mixture was transferred into the 96-well plates seeded with Raji cells which express MHC-II on the surface. Goat anti-mouse IgG Fc-PE antibody was used to detect the binding of LAG-3 protein to Raji cells. The MFI was evaluated by flow cytometry and analyzed by the software FlowJo.

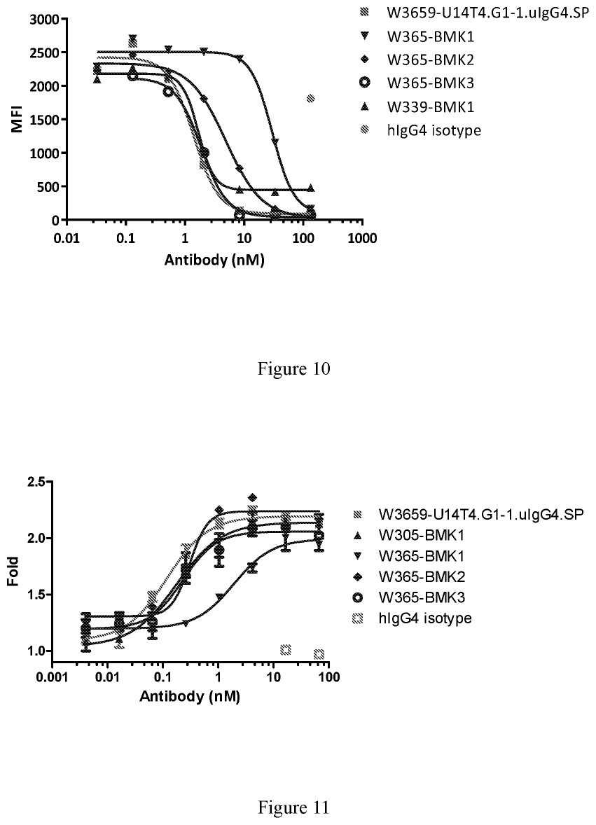

As shown in FIG. 10 and table 13, the EC50 of W3659-U14T4.G1-1.uIgG4.SP for blocking the binding of LAG-3 to MHC-II expressed Raji cells is comparable to W339-BMK1, W365-BMK3 and better than W365-BMK1 and W365-BMK2.

TABLE 13
EC₅₀ of PD-1 × LAG-3 bispecific antibodies
block the binding of LAG-3 to MHC-II

| Antibody | EC₅₀ (nM) |
|---|---|
| W3659-U14T4.G1-1.uIgG4.SP | 1.39 |
| W339-BMK1 | 1.68 |
| W365-BMK1 | 30.0 |
| W365-BMK2 | 4.90 |
| W365-BMK3 | 1.88 |

9. Effects of PD-1×LAG-3 Bispecific Antibodies on PD-1 Expressing Jurkat with NFAT Reporter Gene

Jurkat cells expressing human PD-1 along with stably integrated NFAT luciferase reporter gene and human PD-L1 expressing artificial APC (antigen presenting cell) cells were seeded in 96-well plates. Testing antibodies were added to the cells. The plates were incubated for 6 hours at 37° C., 5% CO₂. After incubation, reconstituted luciferase substrate One-Glo was added and the luciferase intensity was measured by a microplate spectrophotometer.

As demonstrated in FIG. 11, antibodies enhanced NFAT pathway of Jurkat in reporter gene assay. Further, as shown in table 14, the EC₅₀ of W3659-U14T4.G1-1.uIgG4.SP in this assay is better than W365-BMK1 and comparable to other benchmark antibodies.

TABLE 14
EC₅₀ of NFAT pathways enhancement in PD-1 expressing Jurkat.

| Antibody | EC₅₀ (nM) |
|---|---|
| W3659-U14T4.G1-1.uIgG4.SP | 0.12 |
| W305-BMK1 | 0.18 |
| W365-BMK1 | 1.94 |
| W365-BMK2 | 0.31 |
| W365-BMK3 | 0.23 |

10. Effects of PD-1×LAG-3 Bispecific Antibodies on LAG-3 Expressing Jurkat with IL-2 Reporter Gene

Jurkat cells expressing human LAG-3 along with stably integrated IL-2 luciferase reporter gene and Raji cells were seeded in 96-well plates in the presence of SEE (Staphylococcal enterotoxin E). Testing antibodies were added to the cells. The plates were incubated for overnight at 37° C., 5% CO₂. After incubation, reconstituted luciferase substrate One-Glo was added and the luciferase intensity was measured by a microplate spectrophotometer.

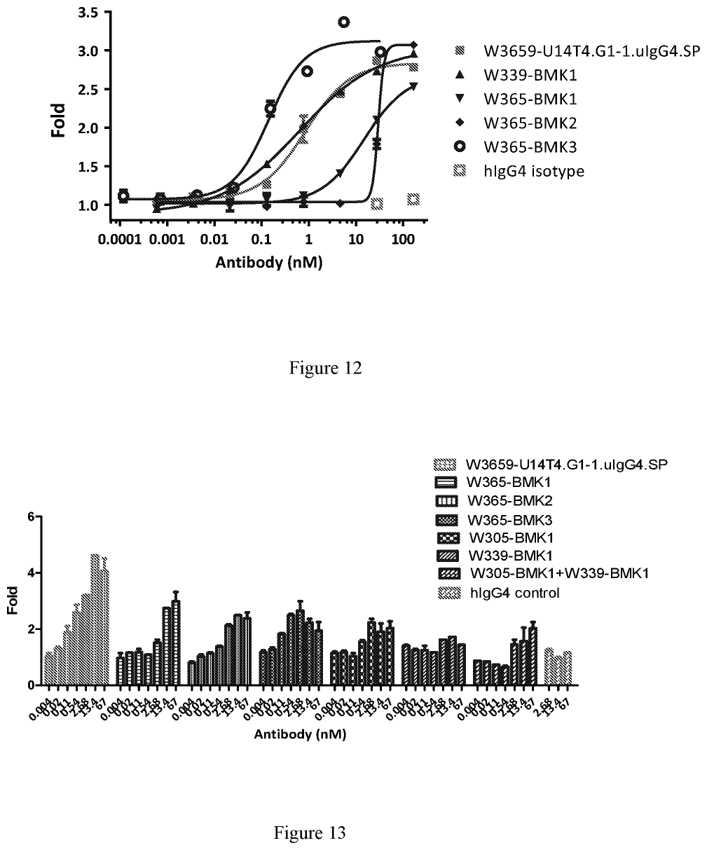

As demonstrated in FIG. 12 and table 15, antibodies enhanced IL-2 pathway of Jurkat in reporter gene assay.

TABLE 15
EC₅₀ of IL-2 pathways enhancement in LAG-3 expressing Jurkat.

| Antibody | EC₅₀ (nM) |
|---|---|
| W3659-U14T4.G1-1.uIgG4.SP | 0.84 |
| W339-BMK1 | 0.65 |
| W365-BMK1 | 14.9 |
| W365-BMK2 | 29.9 |
| W365-BMK3 | 0.14 |

11. Effects of PD-1×LAG-3 Bispecific Antibodies on PD-1 and LAG-3 Expressing Jurkat with NFAT Reporter Gene

Full human LAG-3 plasmid was transiently transfected into Jurkat cells expressing human PD-1 along with stably integrated NFAT luciferase reporter gene. After 48 hours, the cells were seeded in 96-well plates along with PD-L1-expressing Raji cells in the presence of SEE (Staphylococcal enterotoxin E). Testing antibodies were added to the cells. The plates were incubated for overnight at 37° C., 5% CO₂. After incubation, reconstituted luciferase substrate One-Glo was added and the luciferase intensity was measured by a microplate spectrophotometer.

As demonstrated in FIG. 13, antibodies enhanced NFAT pathway of PD-1 and LAG-3 expressing Jurkat in reporter gene assay. The fold is higher than combination of W305-BMK1 and W339-BMK1 as well as other benchmark antibodies.

### 12. Effects of PD-1×LAG-3 Bispecific Antibodies on Human Allogeneic Mixed Lymphocyte Reaction (MLR)

Human peripheral blood mononuclear cells (PBMCs) were freshly isolated from healthy donors using Ficoll-Paque PLUS gradient centrifugation. Monocytes were isolated using human monocyte enrichment kit according to the manufacturer's instructions. Cells were cultured in medium containing GM-CSF and IL-4 for 5 to 7 days to generate dendritic cells (DC). Human CD4⁺ T cells were isolated using human CD4⁺ T cell enrichment kit according to the manufacturer's protocol. Purified CD4⁺ T cells were co-cultured with allogeneic immature DCs (iDCs) in the presence of various concentrations of PD-1×LAG-3 antibodies in 96-well plates. The plates were incubated at 37° C., 5% CO₂. Supernatants were harvested for IL-2 and IFN-γ test at day 3 and day 5, respectively. Human IL-2 and IFN-γ release were measured by ELISA using matched antibody pairs. Recombinant human IL-2 and IFN-γ were used as standards, respectively. The plates were pre-coated with capture antibody specific for human IL-2 or IFN-γ, respectively. After blocking, 50 μL of standards or samples were pipetted into each well and incubated for 2 hours at ambient temperature. Following removal of the unbound substances, the biotin-conjugated detecting antibody specific for corresponding cytokine was added to the wells and incubated for one hour. HRP-streptavidin was then added to the wells for 30 minutes incubation at ambient temperature. The color was developed by dispensing 50 μL of TMB substrate, and then stopped by 50 μL of 2N HCl. The absorbance was read at 450 nM using a Microplate Spectrophotometer.

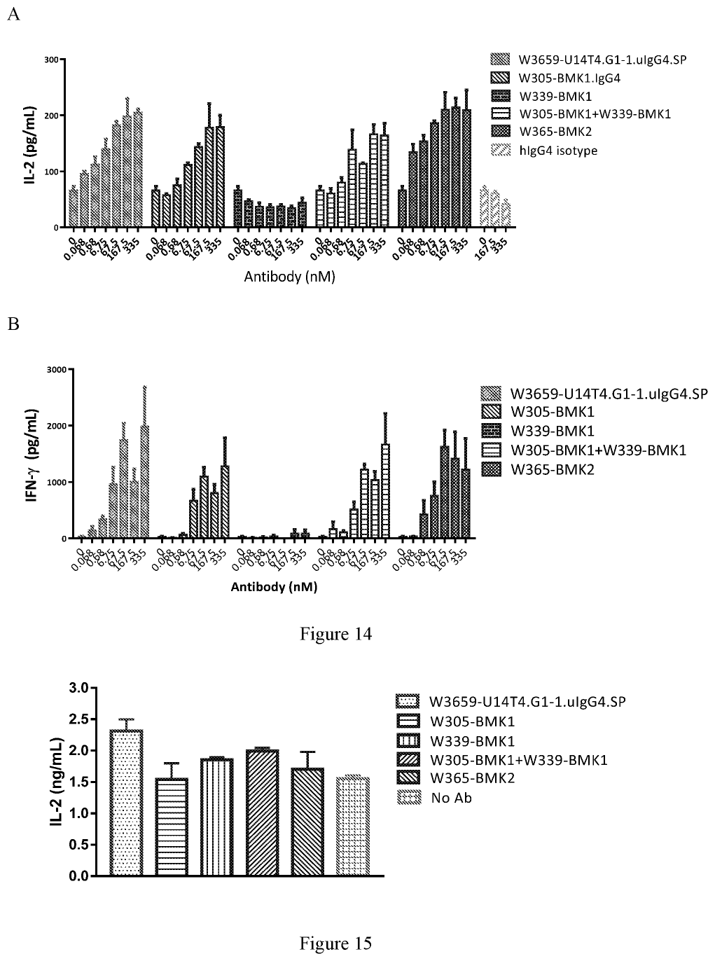

As demonstrated in FIGS. 14A and 14B, W3659-U14T4.G1-1.uIgG4.SP enhanced IL-2 and IFN-γ secretion in mixed lymphocyte reaction.

### 13. Effects of PD-1×LAG-3 Bispecific Antibodies on Human PBMCs Activation

PBMCs and various concentrations of PD-1×LAG-3 antibodies were co-cultured in 96-well plates in the presence of SEB. The plates were incubated at 37° C., 5% CO₂ for 3 days and supernatants were harvested for IL-2 test. Human IL-2 release was measured by ELISA as described in section 12.

As demonstrated in FIG. 15, W3659-U14T4.G1-1.uIgG4.SP enhanced IL-2 secretion in PBMCs stimulated with SEB.

### 14. Thermal Stability Test by Differential Scanning Fluorimetry (DSF)

Tm of antibodies was investigated using QuantStudio™ 7 Flex Real-Time PCR system (Applied Biosystems). 19 μL of antibody solution was mixed with 1 μL of 62.5×SYPRO Orange solution (Invitrogen) and transferred to a 96 well plate. The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tm. If a protein has multiple unfolding transitions, the first two Tm were reported, named as Tm1 and Tm2. Data collection and Tm calculation were conducted automatically by the operation software.

TABLE 16
Tₘ of W3659-U14T4.G1-1.uIgG4.SP in different buffer

| Antibody | pI | Buffer | Tₘ1 (° C.) | Tₘ2 (° C.) |
|---|---|---|---|---|
| W3659-U14T4.G1-1.uIgG4.SP | 6.12 | PBS | 61.0 | ND |
| | | 20 mM Histidine +7% | 64.3 | ND |
| | | Sucrose pH 6.5 | | |
| | | 50 mM NaAC +7% | 63.6 | 69.0 |
| | | Surcose pH 5.6 | | |

### 15. Serum Stability

The lead antibody was incubated in freshly isolated human serum (serum content>95%) at 37° C. At indicated time points, aliquot of serum treated samples were removed from the incubator and snap frozen in liquid N2, and then stored at 80° C. until ready for test. The samples were quickly thawed immediately prior to the stability test.

Plates were coated with mouse Fc-tagged human PD-1 at 1 μg/mL overnight at 4° C. After blocking and washing, various concentrations of PD-1×LAG-3 antibodies were added to the plates and incubated at room temperature for 1 hour after washing. The plates were then washed and subsequently incubated with His-tagged LAG-3 protein for 1 hour. After washing, HRP labeled mouse anti-His antibody was added to the plate and incubated at room temperature for 1 hour. After washing, TMB substrate was added and the color reaction was stopped by 2M HCl. The absorbance at 450 nm was read using a microplate reader.

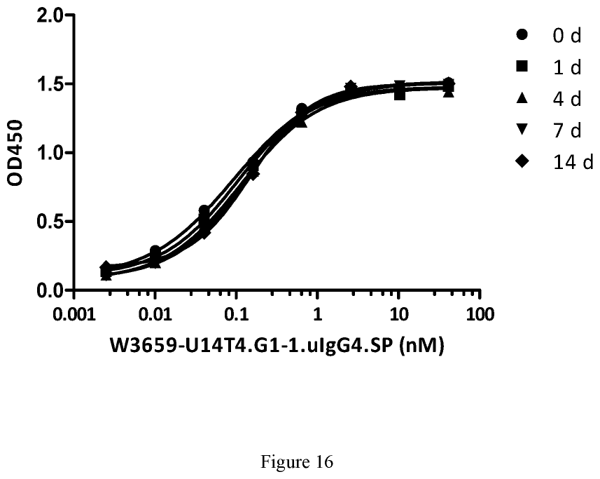

It is demonstrated in FIG. 16 that W3659-U14T4.G1-1.uIgG4.SP was stable in fresh human serum for up to 14 days.

### Example 5: In Vivo Characterization

### 1. In Vivo Anti-Tumor Activity of PD-1×LAG-3 Antibodies

Balb/c mouse (Shanghai Lingchang Biotech) and Colon26 tumor model were used to evaluate the ability of PD-1×LAG-3 antibody to inhibit the growth of tumor cells in vivo. BALB/C mice were implanted subcutaneously with 5×10⁵ mouse colon carcinoma Colon26 cells on day 0 and the mice were grouped (n=8) when the tumor reached 60-70 mm³,

On day 0, day 3, day 7, day 10 and day 14, the mice were intraperitoneally treated with PD-1 mAb (3056) alone (10 mg/kg), LAG-3 mAb (3396) alone (10 mg/kg), PD-1×LAG-3 antibody W3659-U14T4.G1-1.uIgG4.SP (13.9 mg/kg) or combination of 3056 mAb (10 mg/kg) and 3396 mAb (10 mg/kg). Human IgG4 isotype control antibody (10 mg/kg) was given as negative control.

Tumor volume and animal weight were measured for over 3 weeks post-injection. The tumor volume will be expressed in mm³ using the formula: V=0.5ab², where a and b are the long and short diameters of the tumor, respectively.

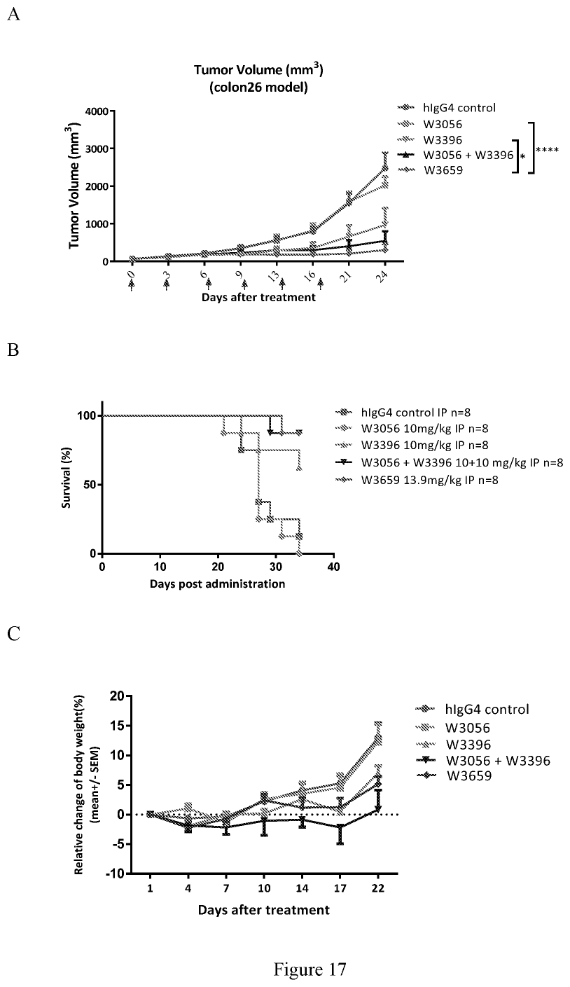

Tumor volume and survival curve of treated mice were shown in FIG. 17A and 17B. The results show that the treatment with W3396 and PD-1×LAG-3 antibody W3659-U14T4.G1-1.uIgG4.SP was effective in Colon26 tumor growth inhibition, while the treatment with the antibody W3056 alone had little effect. W3659-U14T4.G1-1.uIgG4.SP led to greater tumor growth inhibition than the parental PD-1 antibody (W3056) alone or the parental LAG-3 antibody (W3396) alone. The efficacy of W3659-U14T4.G1-1.uIgG4.SP was comparable to combination of PD-1 and LAG-3 antibodies. Meanwhile, in FIG. 17C, the weight growth of each group indicated good safety without obvious toxicity.

1. A bispecific antibody or antigen binding-fragment thereof, comprising a first targeting moiety which specifically binds to PD-1 and a second targeting moiety which specifically binds to LAG-3,
wherein the first targeting moiety comprises a first VHH domain and the second targeting moiety comprises a second VHH domain;
the first VHH domain comprises H-CDR1, H-CDR2 and H-CDR3; wherein the H-CDR3 comprises a sequence as depicted in SEQ ID NO:1, and conservative modifications thereof; the H-CDR2 comprises a sequence as depicted in SEQ ID NO: 2, and conservative modifications thereof; the H-CDR1 comprises a sequence as depicted in SEQ ID NO:3, and conservative modifications thereof;
the second VHH domain comprises H-CDR1, H-CDR2, H-CDR3; wherein the H-CDR3 comprises a sequence as depicted in SEQ ID NO: 4, and conservative modifications thereof; the H-CDR2 comprises a sequence as depicted in SEQ ID NO: 5, and conservative modifications thereof; the H-CDR1 comprises a sequence as depicted in SEQ ID NO: 6, and conservative modifications thereof.

2. The antibody or antigen binding-fragment thereof of claim 1, wherein the first VHH domain comprises a sequence that is at least 70%, 80%, 85%, 90%, 95% or 99% homologous to SEQ ID NO: 7.

3. The antibody or antigen binding-fragment thereof of claim 1 wherein the second VHH domain comprises a sequence that is at least 70%, 80%, 85%, 90%, 95% or 99% homologous to SEQ ID NO: 8.

4. The antibody or antigen binding-fragment thereof of claim 1, wherein the first VHH domain comprises a sequence of SEQ ID NO: 7, and the second VHH domain comprises a sequence of SEQ ID NO: 8.

5. The antibody or antigen binding-fragment thereof of claim 4, wherein the first VHH domain and the second VHH domain are linked by a peptide sequence.

6. The antibody or antigen binding fragment thereof of claim 5, wherein the peptide sequence comprises
(a) an IgG Fc fragment comprising hinge region, CH2 and CH3, and/or
(b) a linker.

7. The antibody or antigen binding fragment thereof of claim 6, wherein the linker comprises a sequence of SEQ ID NO: 9.

8. The antibody or antigen binding fragment thereof of claim 1, comprising a sequence of SEQ ID NO: 10.

9. The antibody or antigen binding fragment thereof of claim 1, wherein the antibody or the antigen binding-fragment
a) binds to human PD-1 with a K_{D} of 2.92E-09 or less; and
b) binds to human LAG-3 with a K_{D} of 3.01E-10 or less.

10. The antibody or antigen binding fragment thereof of claim 1, wherein the antibody is a humanized antibody.

11. A nucleic acid molecule encoding the antibody or antigen binding fragment thereof of claim 1.

12. A cloning or expression vector comprising the nucleic acid molecule of claim 11.

13. A host cell comprising one or more cloning or expression vectors of claim 12.

14. A process for production of the antibody or antigen binding fragment thereof of claim 1, comprising culturing the host cell of claim 13 and isolating the antibody.

15. A pharmaceutical composition comprising the antibody or antigen binding fragment thereof of claim 1, and one or more of a pharmaceutically acceptable excipient, a diluent and a carrier.

16. An immunoconjugate comprising the antibody or antigen binding fragment thereof of claim 1, linked to a therapeutic agent.

17. A pharmaceutical composition comprising the immunoconjugate of claim 16 and one or more of a pharmaceutically acceptable excipient, a diluent and a carrier.

18. A method of modulating an immune response in a subject comprising administering to the subject the antibody or antigen binding fragment thereof of claim 1.

19. (canceled)

20. (canceled)

21. The method of claim 22, wherein the cancer is selected from a group consisting of melanoma, renal cancer, prostate cancer, breast cancer, colon cancer, lung cancer, bone cancer, pancreatic cancer, skin cancer, cancer of the head or neck, cutaneous or intraocular malignant melanoma, uterine cancer, ovarian cancer, and rectal cancer.

22. A method of treating an immune disorder or cancer in a subject comprising administering to the subject a therapeutically effective amount of the antibody or antigen binding fragment thereof of claim 1.
